(19) United States
(12) Patent Application Publication
(10) Pub. No.: US 2012/0328660 A1
(43) Pub. Date: Dec. 27, 2012

(54) IMMUNOGENIC COMPOSITIONS USEFUL IN PROVOKING AN INTEGRATED RESPONSE TO TUMOR ANTIGENS

(76) Inventors: Takemasa TSUJI, New York, NY (US); Sacha Gnjatic, New York, NY (US); Gerd Ritter, New York, NY (US); Lloyd J. Old, New York, NY (US)

(21) Appl. No.: 13/484,884

(22) Filed: May 31, 2012

Related U.S. Application Data
(60) Provisional application No. 61/493,164, filed on Jun. 3, 2011.

(51) Int. Cl. A61K 39/00; A61P 37/04

(52) U.S. Cl. 424/277.1

(57) ABSTRACT

The invention relates to an immunogenic composition and its use. It comprises “OLPs,” or overlapping peptides, derived from a longer, tumor antigen, in combination with polyICLC, and Montanide.

### RELATED APPLICATIONS

This application claims priority from U.S. Provisional Application No. 61/493,164, filed Jun. 3, 2011, incorporated by reference in its entirety.

### FIELD OF THE INVENTION

This invention relates to immunogenic compositions. More particularly, it relates to immunogenic compositions which provoke both a CD4 and a CD8 response to the immunogens in the composition. The composition itself combines Montanide, polyICLC, and at least three peptides which are of a sufficient length such that each peptide may be processed into both a peptide presented by an HLA-Class 1 molecule, and a peptide presented by an HLA-Class II molecule.

### BACKGROUND OF THE INVENTION

A poster presenting at least a portion of this invention was presented at an ASCO meeting during the period of Jun. 4-Jun. 8, 2010.

NY-ESO-1, described in, e.g., U.S. Pat. No. 5,804,381, incorporated by reference herein, is one of the most immunogenic members of the cancer-testis antigen family. It has been shown to be able to induce strong humoral (antibody), and cellular (T cell) immune responses in patients with NY-ESO-1 expressing cancers, either through natural or spontaneous induction by the patients' tumors, or via immunization using defined peptide epitopes. See, e.g., Jager, et al., Proc. Natl. Acad. Sci., USA, 97(22):12198-12203 (2000) and Davis, et al., Proc. Natl. Acad. Sci. USA, 101(29):10697-10702 (2004). An exemplary, but by no means comprehensive, list of references which describe various HLA-Class I and Class II binding epitopes found in NY-ESO-1 includes U.S. Pat. Nos. 7,888,100; 7,619,057; 7,291,335; 7,115,729; 6,723,832; and 6,417,165. For purposes of this application, the sequence of NY-ESO-1 is that set forth in these patents and presented as SEQ ID NO: 1 herein.

More recently, Gnjatic, et al., Adv. Cancer Res., 95:1-30 (2006), incorporated by reference, discuss how NY-ESO-1 has been formulated with different delivery systems and adjuvants. Whereas most compositions designed to stimulate an immune response do so, responses which are strong enough to be useful clinically are limited. It is important, in trying to develop formulations which provoke strong immune responses, to characterize the effect of each component in the formulation, or the nature of the induced response.

It is generally agreed that the most important aim of any immunogenic composition is the induction of reactive CD8⁺ T cells and in the case of compositions useful in treating cancer, developing CD8⁺ T cells which efficiently destroy the tumors.

As was noted supra for NY-ESO-1, but is true for cancer antigens generally, short peptides which satisfy binding motifs for particular HLA-Class I molecules have been used to induce CD8⁺ T cell responses. Most of the responses generated, however, have been of low avidity and the CD8⁺ T cells failed to recognize antigen expressing tumor cells. Further, the use of such short peptides is limited to subjects with the defined HLA-Class I molecule to which the short peptide binds.

Increasing evidence suggests that tumor antigen specific CD4⁺ T cells have important roles in anti-tumor responses, such as the induction and maintenance of tumor reactive CD8⁺ T cells, exerting anti-tumor effects via the secretion of anti-angiogenic cytokines, and also direct cytotoxicity to MHC-Class II expressing tumors. See, e.g., Pardoll, et al., Curr. Opin. Immunol., 10:588-594 (1998); Rakhra, et al., Cancer Cell, 18:485-498 (2010); Nishimura, et al., J. Exp. Med., 190:617-627 (1999); Quezada, et al., J. Exp. Med., 207:637-650 (2010); and van Elsas, et al., J. Exp. Med., 194:481-489 (2001). Recently, Nakanishi, et al., Nature, 462:510-513 (2009) have shown that CD4⁺ T cells are essential to recruiting effector immune cells to the infection site, in a virus infection model in animals. This suggests that tumor antigen specific CD4⁺ T cells have a role in infiltration of other anti-tumor effector cells into tumor sites. Yet another aspect of the role CD4⁺ T cells play involves tumor antigen specific antibodies. The production of such antibodies is mediated by CD4⁺ T cells, and they are considered to enhance CD8+ T cell priming by forming an immune complex which enables cross presentation. See Nagata, et al., Proc. Natl. Acad. Sci. USA, 99:10629-10634 (2002) and Matsuo, et al., Proc. Natl. Acad. Sci. USA, 101:14467-14472 (2004). Hence, it would be desirable to have immunogenic compositions available which induce integrated responses of both CD4⁺ and CD8⁺ T cell responses, plus antibody responses.

Full-length tumor antigens, including, e.g., recombinant proteins, or recombinant viruses which include a coding sequence for such proteins, have been used in such formulations. See, e.g., Jager, et al., Proc. Natl. Acad. Sci. USA, 103:14453-14458 (2006); Davis, et al., Proc. Natl. Acad. Sci. USA, 101:10697-10702 (2004); and Valmori, et al., Proc. Natl. Acad. Sci. USA, 104:8947-8952 (2007). Theoretically, full-length proteins, such as full-length tumor antigens, contain all epitopes for CD4⁺ and CD8⁺ T cells and antibodies, and are applicable to any combination of MHC alleles. Notwithstanding this, the challenges presented by the use of full length proteins are daunting, including mutually exclusive antigen presentation pathways for extracellular and intracellular proteins. Also, as compared to synthetic, single epitope peptides, the manufacture of full length proteins is costly and quality control, including endotoxin levels, is difficult.

Previously, Gnjatic, et al., J. Immunol., 170:1191-1196 (2003), incorporated by reference in its entirety, showed that long peptides (defined infra) are presented efficiently to both CD4⁺ and CD8⁺ T cells by professional APCs (antigen presenting cells), as well as non-professional APCs, like B cells.

Cross presentation of long peptides to CD8⁺ T cells requires proteasomal degradation, which means that the peptides must be internalized in the APCs before loading on HLAs. Bijker, et al., J. Immunol., 179:5033-5040 (2007), have demonstrated that, in a mouse model, long peptides induce CD8⁺ T cells in vivo better than classic short peptides. In this paper, it was shown that ovalbumin specific CD8⁺ T cells were expanded efficiently via immunization with a long peptide containing a CD8⁺ T cell epitope in Incomplete Freund's Adjuvant, while a short peptide induced activated CD8⁺ T cells only transiently. Welters, et al., Canc. Res., 14:178-187 (2008), have shown that efficient induction of CD4⁺ and CD8⁺ T cells specific for human papilloma virus was accomplished using “long overlapping peptides,” or “OLPs.”

It is now accepted that adjuvants which activate innate immune systems, are a critical component of any immunogenic composition. Exemplary adjuvants are toll-like receptor ligands, each of which appears to elicit a different type of response. See, e.g., Akira, et al., Nat. Immunol., 2:675-680 (2001). Such ligands are not without their issues, which include the fact that the expression of toll-like receptors by patients will impact the response. The response also differs from mice to humans, so conclusions from experimental animals are difficult to draw. See, Iwasaki, et al., Nat. Immunol., 5:987-995 (2004). Recent work suggests that ligands such as CpG, imiquimod, and monophosphoryl lipid A, may be useful as adjuvants. See, e.g., Valmori, et al., Proc. Natl. Acad. Sci. USA, 104:8947-9952 (2007); Adams, et al., J. Immunol., 181:776-784 (2008) and Atanackovick, et al., J. Immunol., 172:3289-3296 (2004). Also, Okada, et al., J. Clin. Oncol., 29:330-336 (2011), the disclosure of which is incorporated by reference, used polyinosinic-polycytidylic acid that had been stabilized by lysine and carboxymethyl cellulose, as a maturation agent in a peptide pulsed, human dendritic cell trial. This compound will be referred to as “polyICLC” hereafter. See Sivori, et al., Proc. Natl. Acad. Sci. USA, 101(27):10116-21 (2004), incorporated by reference. Note that poly ICLC should not be confused with poly I:C, i.e., polyinosinic:polycytidylic acid, described as a component of a vaccine by, e.g., Moon, et al., WO 2009/07267 A2; U.S. patent application Ser. No. 12/314,162.

“Cancer-testis” antigens, such as NY-ESO-1, are expressed by a wide range of tumor types, with expression in normal adult tissues being limited to testis. Spontaneous immune responses to, e.g., NY-ESO-1, where subjects are afflicted with NY-ESO-1 presenting peptides, are integrated, i.e., when a spontaneous, anti-NY-ESO-1 antibody response is observed, typically it is associated with NY-ESO-1 specific CD4⁺ and CD8⁺ T cell responses. See, Jager, et al., J. Exp. Med., 187:265-270 (1998); Gnjatic, et al., Proc. Natl. Acad. Sci. USA, 100:8862-8867 (2003). It has also been noted that there is a significant correlation between spontaneous immune responses against NY-ESO-1, and clinical benefit following treatment with anti-CTLA-4 mAbs. See, Yuan, et al., Proc. Natl. Acad. Sci. USA, 105:20410-20415 (2008). There are many reports of experiments where binding peptides from NY-ESO-1, recombinant viruses, DNA vectors, or recombinant full-length protein are administered with and without adjuvants and other delivery systems, and their induced immune responses studied. Exemplary are Jager, et al., Proc. Natl. Acad. Sci. USA, 103:14453-14458 (2006); Davis, et al., Proc. Natl. Acad. Sci. USA, 101:10697-10702 (2004); Valmori, et al., Proc. Natl. Acad. Sci. USA, 104:8947-9952 (2007); Sharma, et al., J. Immunother., 31:849-852 (2008); Odunsi, et al., Proc. Natl. Acad. Sci. USA, 104:12837-12842 (2007); Venaka, et al., Cancer Immun., 7:9 (2007); and Gnjatic, et al., Clin. Canc. Res., 15:2130-2139 (2009).

It has now been found that immunogenic compositions containing at least three OLPs based upon tumor antigens, such as cancer-testis antigens such as NY-ESO-1, in combination with the known substances Montanide (Lee, et al., J. Clin. Oncol., 19(18):3836-47 (2001); Aucouturier, et al., Expert. Rev. Vaccines, 1(1):111-8 (2002), and polyICLC, provoke a strong, integrated immune response, which was surprisingly superior to results secured when the formulations lacked polyICLC, or both polyICLC and Montanide. Both materials are well known to those in the immunological arts. There is little if anything reported, however, on their use together.

The invention is elaborated upon further in the disclosure which follows.

### DETAILED DESCRIPTION OF PREFERRED EMBODIMENTS

### Example 1

Most of the NY-ESO-1 epitopes which have been reported, lie in the central to C-terminus hydrophilic region of the protein. Also the C-terminal and N-terminal ends of the NY-ESO-1 protein have a high homology to LAGE-1. See, e.g., Gnjatic, et al., Adv. Cancer Res., 95:1-30 (2006), incorporated by reference. In view of this, amino acids 1-78 of the protein set forth in SEQ ID NO: 1, as well as amino acids 174-180, were excluded from consideration.

In addition, peptide length was set at 30 amino acids, so as to enable efficient cross presentation. Further, the peptides were designed to present 9 amino acid overlaps, to maximize the number of potential CD4⁺ and CD8⁺ epitopes available for presentation and recognition. One final consideration was to exclude any peptides which possessed strong anchor HLA binding motifs at their C terminus, in order to avoid generation of cryptic epitopes. See, Gnjatic, et al., Proc. Natl. Acad. Sci. USA, 99:11813-11818 (2002), incorporated by reference.

The result of these considerations was the design of 4 peptides consisting of amino acids 79-108, 100-129, 121-150, and 142-173 of SEQ ID NO: 1, which were synthesized via known methods and formulated as lyophilized powder in 25 mg palmitoyl oleoyl phosphatidyl choline (POPC). Hereafter, any reference to “the peptides” refers to a mix of equal amounts of these 4 peptides. They will also be referred to as the “OLPs,” or “overlapping long peptides.” The first and fourth of these peptides are disclosed in Old, et al., U.S. Pat. No. 7,259,235, incorporated by reference in its entirety.

A “library” of NY-ESO peptides was also obtained, which consisted, for the most part, of 20-mers, with 10 amino acid overlaps. It consisted of three distinct pools of 20 mers, with 10 amino acid overlaps, as stated supra. The pools corresponded to amino acids 1-80, 71-130, and 119-180 of SEQ ID NO: 1. This library served in the analysis of T cell responses, reported infra, to eliminate detection of responses to impurities, and was also used as the “assay OLPs” as referred to infra.

### Example 2

A total of 28 patients having Stage 11 to IV histologically documented epithelial carcinoma arising in the ovary, fallopian tube, or peritoneum were chosen for the study. Patients had initially received cytoreductive surgery and platinum/taxane based chemotherapy. Following relapse, they returned to a second or third complete clinical remission following additional chemotherapy. Median time from the end of chemotherapy to the first immunization was 2.8 months, with a range of 1.2 to 5.5 months. Remission was defined as CA 125<35 U/mL, an unremarkable physical examination, and no definite evidence of disease by computer tomography. Nonspecific lymph nodes or soft tissue abnormalities <1 cm were permitted. Cytotoxic chemotherapy was to have concluded at least 4 weeks before the start of the study. The patients were sequentially enrolled in 3 cohorts. Patients received at least 1 subcutaneous immunization in rotating sites on the upper arms regardless of the expression of NY-ESO-1 in tumor tissues. Patients Patents in Cohort 1 received 1.0 mg NY-ESO-1 OLPs in 0.5 mL diluent; Cohort 2 received 1.0 mg NY-ESO-1 OLPs in 0.5 mL diluent+0.5 mL Montanide-ISA-51 VG (total of 1.0 mL); Cohort 3 received 1.0 mg NY-ESO-1 OLPs in 0.3 mL diluent+0.7 mL (1.4 mg) Poly-ICLC+1.0 mL Monatinde-ISA-51 VG (total of 2.0 mL administered in two syringes containing 1.0 mL each). The compositions were administered on weeks 1, 4, 7, 10, and 13 with final study safety assessment on week 16. DTH testing was performed with 1 mg lyophilized NY-ESO-1 OLPs at pre-treatment and at week 16. From 24 patients with available tumor specimens, 2 had strong and 7 had focal (<5% sample) NY-ESO-1 expression by immunohistochemistry. Blood was taken from each patient at pretreatment, before each administration and at week 16, for research studies. The ability to complete all 5 vaccinations was a total of 3/4 in Cohort 1, 8/13 in Cohort 2, and 5/11 in Cohort 3 completed all five immunizations. The main reasons for study discontinuation were progressive disease (1/4 in Cohort 1, 2/13 in Cohort 2, and 1/11 in Cohort 3) and early study closure (4/11 in Cohort 3). A total of 20 patients received all injections, as some patients left the study as a result of various conditions. Such is not unexpected in studies of this type.

### Example 3

Assays were carried out on plasma samples which were obtained from the whole blood samples referred to supra. Blood samples were taken before the start of the treatment, before each immunization, and one more time following the last immunization. To obtain the plasma, whole blood was centrifuged following standard techniques, and then the plasma was stored, at −80° C. until it was used. ELISAs were carried out in accordance with Gnjatic, et al., Methods Mol. Biol., 520:11-19 (2009), incorporated by reference, using recombinant tumor antigen proteins NY-ESO-1, LAGE-1, MAGE-A1, MAGE-A3, and p53, and DHFR as a control. In some experiments, assay OLPs were used as the coating antigen. A reciprocal titer was estimated from optical density readings of serially diluted plasma samples. Negative control sera from healthy individual and positive control sera for each antigen from cancer patients were always included in all assays. The anti-human immunoglobulin antibodies used as secondary reagents were alkaline phosphatase (AP)-labeled goat-anti-human IgG, biotinylated mouse-anti-human IgG1, AP-labeled mouse anti-human IgG2, AP-labeled mouse anti-human IgG3, AP-labeled mouse anti-human IgG4, AP-labeled goat-anti-human IgA, AP-labeled mouse-anti-human IgD-AP, AP-labeled goat-anti-human IgE, and AP-labeled goat-anti-human IgM. Reciprocal antibody titers by ELISA were considered significant if >100.

One of the subjects exhibited significant, spontaneous anti-NY-ESO-1 antibody production before immunization. Patients who received the mixture of the OLP, and polyICLC in Montanide developed humoral responses sooner than patients who did not receive polyICLC. Specifically by week 7, NY-ESO-1 specific IgG were measured in 6/12 patients who received the OLP and Montanide compared to 9/10 patients who received OLP, polyICLC, and Montanide, whereas none of the patients who only received only OLP showed any significant response at 7 weeks except the baseline seropositive patient. The patients who received OLP and Montanide did in fact develop an immune response at a later point in time, and with an average titer that was significantly lower than those who received the OLP and polyICLC in Montanide.

As was noted, supra, humoral responses to LAGE-1, MAGE-A1, MAGE-A3, and p53 were also measured. All of these tumor antigens are known to be expressed by ovarian tumors. Eight of 28 patients showed a significant, pre-existing anti-p53 IgG response. Some patients developed low titer IgG responses to MAGE-A1 and MAGE-A3 indicating potential antigen spreading by vaccination.

### Example 4

In these experiments, the epitopes recognized by the NY-ESO-1 specific IgGs that were induced by immunization were determined. ELISAs were carried out, using standard methods, with the assay OLPs supra being used as coating antigens. Antibody titers of subjects were tested at week 16, with the exception of one sample, which was tested at week 13. IgG responses were most frequent and strongest against the NY-ESO-1 region consisting of amino acids 121-150. The NY-ESO-1 region consisting of amino acids 80-109 was also recognized frequently, and in the case of the NY-ESO-1 region consisting of amino acids 100-129, all patients who received the combination of the peptides, Montanide, and polyICLC had relevant antibodies, as did 4/9 of the patients who received the peptides in Montanide only. There was a much lower response to the NY-ESO-1 region consisting of amino acids 142-173. In all cases, polyICLC enhanced the humoral, immune response, not only by accelerating the response and increasing titer, but also by broadening antibody repertoire.

### Example 5

It is well known and accepted that the induction of antigen specific IgG as well as other immunoglobulin responses, is mediated by CD4⁺ T cells. Further, various cytokines, which are produced by CD4⁺ cells are thought to play a role in “class switching” of antibodies, which in turn results in different patterns of immunoglobulin isotopes.

The antibody response to the immunogenic compositions described supra was analyzed via ELISA, using different isotype specific monoclonal antibodies.

It was observed that most of the antibodies generated were IgG1 and IgG3, which activate complement and bind FcγR,

Only three patients showed any significant IgG2 response, while only one patient showed significant IgG4 response. There were also two patients who showed an IgM response, and two with an IgA response.

### Example 6

In this example and the examples which follow, the T cell responses of the immunized patients was studied more closely.

First, peripheral blood mononuclear cells (PBMCs) were isolated by centrifugation over Lymphocyte Separation Medium, using standard methods, and were then stored in liquid nitrogen.

Next, CD4⁺ and CD8⁺ T cells were separated from the PBMCs using magnetic beads coated with relevant T cell antibodies. Then, the two groups (CD4⁺ and CD8⁺ cells) were stimulated, independently with T cell depleted cells, which had been pulsed, overnight, with the assay OLPs at either 6 μ/M per peptide, or 100 nM per peptide. Presensitization with adenovirus recombinant for NY-ESO-1 (1000/IU cell) was also performed for most patients but only for some time points because of the limitation in the number of PBMC available.

The separated populations were cultured in the presence of 10 U/ml IL-2, 20 ng/ml IL-7 in RPMI, supplemented with 10% human AB serum, 2 mM L-glutamate, 100 U/ml penicillin, 100 ug/ml streptomycin, and 1% non-essential amino acids.

Next, the number of IFN-γ producing, NY-ESO-1 specific T cells was evaluated by an ELISPOT assay at 9-14 days post culture (for CD8⁺ cells) or 19-23 days (for CD4⁺ cells). The ELISPOT assay has been described by Atanackovic, et al., Proc. Natl. Acad. Sci. USA, 105:1650-55 (2008), incorporated by reference. To elaborate, nitrocellulose coated microtiter plates were coated, overnight, with an anti-IFN-γ monoclonal antibody (2 μg/ml) and blocked with 10% human serum, in RPMI 1640 medium.

Additional assays were conducted with autologous, Epstein Barr virus transformed B cell lines (“EBV-B” cells, hereafter), which were generated from supernatant produced by B95-8 cells, in RPMI+10% fetal calf serum.

The EBV-B cells described supra, were pulsed with 10 μM of the assay OLPs, or infected with vaccinia virus which encoded either NY-ESO-1, or influenza virus nucleoprotein in order to create target cells for an ELISPOT assay. These processes were carried out, overnight, at 37° C.

Following this, varying sized samples of effector cells were co-cultured with 5×10⁴ antigen pulsed EBV-B cells, for 24 hours in RPMI without serum.

The plates were then developed using 0.2 μg/ml biotinylated, anti-IFN-γ mAb, 1 U/ml streptavidin—alkaline phosphatase conjugate, and 5-bromo-4-chloro-3-indolyl phosphate/nitroblue tetrazolium. Spots were evaluated using standard methods. Results were taken in terms of the average number of spots from duplicate wells, without subtracting background spots, against unpulsed target. An antigen specific IFN-γ response, with a spot count 3 times more than background spots obtained with non-pulsed targets, was deemed significant.

The results indicated that CD8⁺ cells taken from patients immunized only with peptides produced almost no IFN-γ, except for the baseline seropositive patients referred to supra. Of 13 patients immunized with peptides in Montanide, 8 showed sporadic or weak and transient responses. The exception was patient who showed a preexisting sustained response. In contrast, the CD8⁺ cells from 10 patients of 11 who received the peptides, Montanide and polyICLC exhibited a response, even after only a single immunization. In 7 patients the response was consistent and sustained after vaccination.

The results set forth supra were confirmed, in assays using tetrameric complexes of HLA class I molecules, loaded with a relevant peptide. In brief, CD8⁺ T cells were stimulated, once, with T cell depleted PBMCs that had been pulsed with the peptides described supra.

These CD8⁺ T cells were cultured in the manner described supra for 10 days and were then contacted to tetramers of HLA-Cw*03 and a peptide consisting of amino acids 92-100 of SEQ ID NO: 1 (a known binder to HLA-Cw*03), and tetrameric complexes of HLA-A*02 and the peptide consisting of amino acids 157-165, also a known HLA binder. The staining results which were positive validated the earlier experiments.

### Example 7

Studies were carried out to determine if immunization with long peptides of the type described herein would result in CD8⁺ T cells with high avidity which also recognize naturally processed NY-ESO-1 peptides.

In a first set of experiments, CD8⁺ T cells were presensitized with T cell depleted PBMCs which had been transfected with adenoviral vectors expressing NY-ESO-1. Adenoviral transfection was done according to Gnjatic, et al., Proc. Natl. Acad. Sci. USA, 97:10917-22 (2000)). Briefly, a million cells were mixed with adenovirus with an infection rate of 1000 IU/cell. The cells were incubated over night at 37° C. in 5% CO₂, then washed twice before use.

CD8⁺ T cells were obtained, as described supra, and were stimulated, once, with the PBMCs that were infected with NY-ESO-1 producing adenovirus vector. Methodologies for doing this are well known in the art and need not be repeated here.

Ten days following stimulation, IFN-γ producing CD8⁺ T cells were enumerated using the ELISPOT assay described supra.

The adenovirus infected cells induced expansion of CD8⁺ T cells which responded to naturally processed intracellular NY-ESO-1. This did not always occur in patients who had a significant CD8⁺ T cell response after peptide presensitization, which showed that the immunization also induced low avidity, CD8⁺ T cells.

### Example 8

In addition to the experiments set forth in the previous example, CD8⁺ T cells specific for NY-ESO-1 were tested for their recognition of target cells infected with Vaccinia virus encoding NY-ESO-1.

To carry this out, CD8⁺ T cells were stimulated once with T cell depleted PBMCs which had been pulsed with assay OLPs, using standard methods. These CD8⁺ T cells were then contacted to target cells, which were autologous EBV-B cells that had been infected with NY-ESO-1 encoding Vaccinia virus, or influenza virus nucleoprotein. The contact was made 10-13 days after stimulation. The IFN-γ producing CD8⁺ T cells were enumerated using the ELISPOT assay described supra.

Prior work had shown that spontaneously induced NY-ESO-1 specific CD8⁺ T cells in seropositive cancer patients recognize both NY-ESO-1 expressing tumors and vaccinia virus induced NY-ESO-1 efficiently. This was observed herein, where the T cells were able to recognize target cells infected with vaccinia virus encoding full-length NY-ESO-1 (vvESO) in 4/16 patients tested after vaccination, including the baseline NY-ESO-1-seropositive patient. Absence of vvESO recognition in some patients could be ascribed to the expansion of low-avidity T cells after in vitro presensitization with the regular 6 μM of the assay OLP.

It was believed possible that the presensitization with high concentrations of the peptide expanded low avidity CD8⁺ T cells, leading to obscuring of low frequency, high avidity CD8⁺ T cells.

In order to test this hypothesis, CD8⁺ T cells were taken from a patient sample at the tenth week of the immunization protocol. These were then presensitized, once, with T cell depleted PBMCs which had been pulsed with either 6 μM or 100 nM of the peptides. Any CD8⁺ T cells which produced IFN-γ in an ELISPOT assay were isolated after they had been restimulated with antigen presenting cells which naturally presented NY-ESO-1 peptides on their surfaces, and then polyclonally expanded, followed by testing again to determine if they recognized the vaccinia produced NY-ESO-1 protein.

Significant expansion of vaccinia virus induced, reactive CD8⁺ T cells was observed following presensitization with the low dose of OLPs indicating that, indeed, high avidity NY-ESO-1 specific CD8⁺ T cells were induced.

### Example 9

As noted, supra, both CD8⁺ and CD4⁺ cells were separated from the patient samples. The preceding examples detailed studies on the CD8⁺ population. The examples which follow discuss experiments with the CD4⁺ cells.

Briefly, CD4⁺ cells were presensitized as described supra with assay OLP covering all of NY-ESO-1 and tested against EBV-transformed B cells pulsed with three assay OLP subpools representing NY-ESO-1 aa 1-80, NY-ESO-1 aa 71-130 and NY-ESO-1 aa 119-180.

After 19-23 days of culture, the CD4⁺ cells were tested for IFN-γ production, and any positive cells were evaluated via ELISPOT assay.

As with the CD8⁺ cells, the induction of CD4⁺ cells was enhanced significantly by Montanide, and even more so with the combination of Montanide and polyICLC.

### Example 10

The experiments described supra revealed that immunization using Montanide and polyICLC plus the peptides enhanced the peptides' immunogenicity greatly. In these experiments, a more in depth characterization of the CD4⁺ T cell induced response was pursued.

Previously, it was reported that by staining for CD154 after antigenic restimulation, low frequency CD4⁺ cells could be isolated and analyzed after polyclonal expansion. See, Tsuji, et al., J. Immunol., 183:4800-4808 (2009), incorporated by reference. Briefly, presensitized CD4⁺ T cells were restimulated for 6 hours with the equal number of APCs that had been pulsed overnight with assay OLP and labeled with CFSE, in the presence of 20 μl of PE-conjugated anti-CD154 mAb and 0.3 μl GolgiStop. Unpulsed APCs were used as a negative control. CFSE⁻CD154⁺ NY-ESO-1-specific CD4⁺ T cells were sorted by a FACSAria instrument and FACSDiva software.

The method described by Tsuji, et al., supra, was used to analyze samples taken from patients before and after immunization. Essentially, the protocols for stimulating supra, were followed, and cells were assayed for expression of CD154. CD154⁺ T cells were detectable both before and after the immunization. The CD154⁺ T cell background were less than 2%. There was a significant difference in the kinetics of induction of CD4⁺ T cells via immunization. Immunization with the combination of Montanide/polyICLC and peptides significantly accelerated induction of responses, resulting in a higher frequency of CD154⁺, NY-ESO-1 specific T cells as compared to subjects who did not receive polyICLC.

### Example 11

It has been observed (see, e.g., Welters, et al., Cancer Res., 14: 178-187 (2008); Giannopoulos, et al., Leukemia, 24:798-805 (2010), that immunization may expand immunosuppressive regulatory T cells (“Treg” hereafter). Nishikawa, et al., Blood, 106:1008-1011 (2005); and Nishikawa, et al., J. Immunol., 176:6340-6346 (2006), have shown that Treg suppress in vitro expansion of naïve NY-ESO-1 specific CD4⁺ cells.

In order to investigate the effect of Treg on the expansion of the CD4⁺ cells under consideration, any CD25⁺ T cells, which included the Tregs, were removed from the population via known methods, to create a CD4⁺CD25⁻ population, which was then stimulated, once, with T cell depleted PBMCs that had been pulsed with the assay OLP described supra. After 20 days of culture, the cells were tested in ELISPOT assays, as described supra. EBV-B cells pulsed with assay OLP pools representing NY-ESO-1 aa 71-130 or NY-ESO-1 aa 119-180 were used as target cells in the ELISPOT assay. Whole CD4⁺ T cells were used as control and treated as described supra.

The results confirmed prior findings, i.e., NY-ESO-1 specific CD4⁺ T cells which had not been detectable in the whole CD4⁺ T cell population become detectable in some, depleted samples. In contrast to pre-vaccination samples the effect of the removal of CD25⁺ T cells at week 4, 13, and 16 was not consistent, increasing in some samples and decreasing in others, or having no effect at all. This indicates that it is unlikely that vaccination systematically induced NY-ESO-1 specific CD25⁺ Tregs able to actively suppress the expansion of NY-ESO-1 specific CD4⁺ effector T cells.

The experiments described supra revealed that immunization using Montanide and polyICLC plus the OLPs enhanced the peptides' immunogenicity greatly. In these experiments, a more in depth characterization of the CD4⁺ T cell induced response was pursued.

In further experiments, CD154⁺ cells were isolated via flow cytometric sorting, and stimulated with 10 μg/ml PHA in the presence of irradiated PBMCs. Following stimulation, the cells were expanded, for about 20 days, in RPMI and 10% SAB, in the presence of 10 U/ml IL-2, and 20 ng/ml of IL-7 in order to create CD4⁺ T cell lines. Epitopes recognized by the cells lines were determined by stimulating CD4⁺ T cell lines (50,000 cells) from before vaccination, at week 13 or 16 with autologous EBV-B cells (50,000 cells) pulsed or unpulsed with a single assay OLP and 24 hours later, supernatant was harvested and was evaluated for GM-CSF levels by ELISA. The epitopes were widely distributed over the hydrophobic regions covered by the OLPs used for immunization.

It was of interest to observe that, although the first 78 N terminus amino acids of NY-ESO-1 were not used in the immunizations, 3 of the patients showed a consistent response to epitopes from this region. These were all samples from patients who had been immunized with peptides, Montanide, and polyICLC.

### Example 13

The pattern of cytokine production by the induced CD4⁺ T cells was studied next.

Equal amounts of CD4⁺ T cell lines created supra, and autologous EBV-B cells (5×10⁴) were co-cultured in 250 μl RPMI and 10% FCS. After the EBV-B cells had been pulsed overnight or not with the assay OLPs and cultured for 24 hours, supernatant was harvested, and stored at −20° C. until sandwich ELISAs were carried out using standard methods. The cytokines which were assayed included IFN-γ, GM-CSF, IL-4, IL-9, IL-10, IL-13, IL-17, and TGF-β. These are all known to be produced by different CD4⁺ subsets.

All CD4⁺ T cells produced significant amounts of GM-CSF. The levels differed, however, which indicated that the purity of the samples differed.

Most of the cell lines produced significant, but varying levels of IFN-γ, IL-4, IL-10, and IL-13. Only a few samples were positive for IL-17 and TGF-β. This indicates that the immunization induced Th1 and Th2 type responses, but not Th17. The ratio of IFN-γ/IL-4 was significantly higher in the T cells isolated from subjects who received the 3-part composition, as compared to those who did not receive polyICLC. The levels of IL-4, IL-9, and IL-13 were significantly suppressed by polyICLC at week 13 and 16, which is consistent with reduced Th2 and Th9 responses and enhanced Th1 differentiation.

Production of TGF-β was not detected consistently in any of the cells examined, which indicated that TGF-β producing regulatory T cells were either not induced via the immunizations, or they did not expand when stimulated in vitro.

It was of interest that small, but significant, amounts of IL-9 were generated by T cells following immunization with OLPs alone, or OLPs, with Montanide when polyICLC was added to either of these formulations, IL-9 production was inhibited completely. Also significant is the fact that some but not all IL-9 producing cells also produced IL-4. It appears that the inhibition of Th9 by the polyICLC was not simply due to downregulation of Th2 responses, as the IL-9/IL-4 ratio was significantly lower when polyICLC was used.

### Example 14

Nishikawa, et al., J. Immunol., 176:6340-6346 (2006) have shown that immunization with peptides can elicit low avidity CD4⁺ T cells that do not recognize naturally processed exogenous proteins. Given this, it was important to investigate the ability of the CD4⁺ T cells described herein, to recognize NY-ESO-1 proteins.

When CD4⁺ T cell lines were isolated from the subjects before vaccination and tested for their ability to recognize NY-ESO-1, it was found that many of the cells recognized NY-ESO-1 proteins significantly, indicating that most subjects had high avidity NY-ESO-1-specific CD4⁺ T cell precursors although their frequency was extremely low. Interestingly this property was decreased following immunization with the peptides alone. In contrast immunization using the three-part formulation increased the ability of the CD4⁺ T cells to recognize peptides very sharply.

In order to investigate the underlying mechanism, equal numbers of autologous CD4⁺ T and EBV-B cells (5×10⁴) were combined, where the EBV-B cells had been pulsed with graded concentrations of assay OLPs or not pulsed. Twenty-four hours later, supernatant was collected, and tested for GM-CSF via ELISA. Apparent avidity (EC₅₀) was defined as the peptide concentration required to induce 50% of GM-CSF levels against 10 μM peptide after interpolation of fitting curves. EC₅₀ was determined at pretesting, week 7 and week 13. Functional avidities increased, and the avidities of patients immunized with the two, and three part formulations was substantially higher than the CD4⁺ T cells from patients immunized with OLPs alone.

Emulsifying OLPs in Montanide appeared to expand high avidity CD4⁺ T cell populations efficiently, as compared to the results with OLP only. While expansion of low avidity CD4⁺ T cell populations could be the reason for this, the fact that immunization with peptides alone did not expand the NY-ESO-1 specific CD4⁺ T cells (see, supra), suggests that high avidity T cells are being deleted.

The foregoing disclosure sets forth features of the invention, which is an immunogenic composition comprising at least three “long” peptides, as explained herein, in combination with Montanide and polyICLC. The compositions are useful in generating a coordinated immune response, which includes a CD4⁺ and CD8⁺ T cell response. In especially preferred embodiments, the long peptides are peptides consisting of amino acid sequences found in a cancer associated antigen, such as a cancer testis antigen, described supra. While NY-ESO-1 is a preferred embodiment of such cancer testis antigens, the artisan of ordinary skill will be familiar with other members of the families of cancer testis, and cancer antigens, and these will not be set forth herein.

“Long peptides” as used herein means that the selected peptides will be long enough to include at least one CD8⁺ specific epitope, and one CD4⁺ specific epitope. In practical terms, this means the peptides will be at least 30 amino acids in length, and may be as long as 50 amino acids. Preferably, the peptides are selected so that there is overlap in sequence amongst them. Specifically a first and second peptide are chosen so that the C-terminal 9-15 amino acids of the first peptide are the same as the N terminal 9-15 amino acids of the second peptide, and the third amino acid's N-terminal 9-15 amino acids overlap the C-terminal amino acids of the second peptide, and so forth.

The selection of peptides can be based on known binding motifs for different HLA Class I and II molecules following, e.g., Marsh, et al., The HLA Factsbook (Academic Press 2000), incorporated by reference or the information provided at www.syfpethi.de, or Immunogenics, 50:213-219 (1999), incorporated by reference.

The examples, supra, used three overlapping long peptides from NY-ESO-1. It should be borne in mind that combinations of one or two of these, without the third peptide, plus one or more long peptides, either from the NY-ESO-1 sequence, or other cancer antigens, may be used. The skilled artisan will base the formulations on the cancer antigens presented by tumors of a particular subject, as well as the particular HLA molecules expressed by that subject. The overlapping peptides can be chosen to occur in one, or more than one, tumor antigen. Exemplary of such antigens in addition to NY-ESO-1 are MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, LAGE-1, SSX-1, SSX-2(HOM-MEL-40), SSX-1, SSX-4, SSX-5, SCP-1 or CT-7. The choice of OLPs can, but need not be made based upon which tumor antigens are expressed by the patient. The 4 OLPs chosen in the prior examples were designed to embrace approximately 90% of all CD4 and CD8 epitopes, and other formulations, based upon other antigens, are within the ken of the skilled artisan.

The immunogenic compositions comprise at least 2, preferably at least 3, and most preferably, at least 4 different OLPs, and may include up to 12, and preferably no more than ten different OLPs. Preferably, each peptide consists of from 25-50 amino acids, more preferably, 30-45, and most preferably 30-40 amino acids.

Formulations can be manufactured which include different quanitites of each of the active ingredients. In the case of peptides, a total of from about 0.1 to about 5 mg, more preferably about 0.5 to about 2.5 mg, and most preferably from about 0.5 to about 2.0 mg of peptide are present in each dose. The different peptides should be present in equal amounts. For example, in the examples presented herein, a total of 1 mg of peptide was used, divided equally as 0.25 mg of each peptide.

Montanide is present in an amount ranging from about 0.1 mL to about 2.0 mL per dose, preferably 0.1 mL to about 5 mL per dose, and most preferably, from about 0.5 mL to about 1 mL per dose. The examples, supra, used either a 0.5 mL or a 1.0 mL dose of Montamide.

The polyICLC is used in an amount ranging from about 0.5 mg to about 4.0 mg per dose, more preferably from about 0.5 mg to about 2.5 mg per dose, and most preferably, from about 1.0 mg to about 2.0 mg per dose. An amount of 1.4 mg of polyICLC was used in the experiments, supra.

Other features of the invention will be clear to the skilled artisan and are not set forth herein.

The terms and expression which have been employed are used as terms of description and not of limitation, and there is no intention in the use of such terms and expression of excluding any equivalents of the features shown and described or portions thereof, it being recognized that various modifications are possible within the scope of the invention.

We claim:

1. An immunogenic composition comprising:
(i) Montanide;
(ii) polyICLC, and
(iii) at least three peptides consisting of amino acid sequences found in a tumor antigen, wherein each of said peptides;
(a) contains an epitope for binding to an HLA-Class I molecule,
(b) contains an epitope for binding to an HLA-Class II molecule,
(c) contains at least 25 and no more than 50 amino acids, and
(d) each of said peptides amino acid sequences overlaps the amino acid sequence of another peptide in said immunogenic composition, by from 9 to 15 amino acids.

2. The immunogenic composition of claim 1, wherein said tumor antigen is a cancer testis antigen.

3. The immunogenic composition of claim 2, wherein said cancer testis antigen is NY-ESO-1 having the amino acid sequence of SEQ ID NO: 1.

4. A method for generating an integrated immune response to a tumor antigen comprising administering the immunogenic composition of claim 1 to a subject who has a tumor which expresses said tumor antigen.


SEQUENCE LISTING (1 sequence)

SEQ ID NO: 1 - protein, 180 aa, Homo sapiens
MQAEGRGTGGSTGDADGPGGPGIPDGPGGNAGGPGEAGATGGRAPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR